(19) United States
(12) Patent Application Publication
(10) Pub. No.: US 2013/0244319 A1
(43) Pub. Date: Sep. 19, 2013

(54) COMPREHENSIVE SYSTEM AND METHOD FOR PRODUCING ALGAE

(71) Applicant: Geronimos Dimitrelos, Oakland Park, FL (US)

(72) Inventor: Geronimos Dimitrelos, Oakland Park, FL (US)

(21) Appl. No.: 13/423,735

(22) Filed: Mar. 19, 2012

(51) Int. Cl. C12M 1/42

(52) U.S. Cl. 435/292.1

(57) ABSTRACT

A system of growing and harvesting algae is provided whereby the system encompasses a plurality of controlled growth and stress steps to optimize growth rate versus time.

### BACKGROUND OF THE INVENTION

Many aspects of our current consumer and producer driven society have developed a perception of a need for renewable and sustainable resources. Many companies have turned to the use of algae to assist in this endeavor. Algae can be used in the food chain whereby algae is either grown as a food substance or given to animals within the food chain. Algae can also be used in the farming industry to support farmers by providing a renewable source of fertilizer. Additionally, algae can be used as an alternative to petroleum products, in the polymer and plastics industry, cosmetic industry, paint and die industry, nutraceuticals, pharmaceuticals etc. There are many known processes for growing and harvesting algae. However, many of these processes take significant amount of time that do not provide for economic feasibility. It is therefore desired that a process be developed that optimizes growth and harvest time such that economic feasibility can be achieved.

### SUMMARY OF THE INVENTION

In one embodiment of the present invention, manufacturing and production process is a 3 Phase activity:
Grow the Algae
Harvest Enhancement
Mechanical Separation

### Phase I: Grow the Algae

The algae will grow in two phases: exponential and plateau. These names relate to the rate of cell division as a function of time. Some algae or growth regiments grow their algae at a slower pace. This can be interpreted on a two dimensional graph (X vs. Y) as a linear process. When the algae are initially growing they grow in exponential phase. In simplest terms, their cells are dividing at a much faster rate than compared to linear growth. In plateau phase, as cell division rate is reduced, the physiochemical growth medium is modified so the algae maximize the intracellular production of the compounds just before harvest. This is prior to harvest only and is important because the plateau phase induces stress on the algae such that continued exposure to this stress regime would cause the cells to degrade. By exposing cells to this phase just before harvest and only just before harvest, it maximizes the cell density and the concentration of the compounds in the algae.

The algae are grown in a step-wise manner from lab controlled cultures into large environmentally controlled 11,355 liter vessels. These systems are generically termed photobioreactors (PBR). The system and method of the present invention utilizes a multitude of PBR's depending on the volume of the culture during their exponential growth phase. The invention uses different types of PBR's to maximize growth rate based on the physical delivery capabilities of nutrients, light and gas exchange across the air/water interface. The process only changes vessels when the cell density to delivery capability ratio can be maximized through the use of a different PBR.

The present invention also uses a combination of multi-phasic LED light sources combined with natural day lighting and fiber optic driven daylight. It has previously been shown through peer reviewed journals that strobing biological cells can induce growth changes. The present invention has discovered a unique combination to use the multi-phasic capabilities of sophisticated LEDs to optimize the growth rate of the algae. On average, the lighting schedules will be 16 hours of light and 8 hours of darkness. This process allows the process to out produce other known open-pond and even other closed-loop PBR cell density production numbers on a time versus time basis. Put more simply, these algae to grow faster than others.

The invention optimizes the nutrient load from a biological response as a function of multiple ion concentration interactions. The majority of past research and current optimization relies on a 2 or 3 ion response reaction. In this way either 2 or 3 ions are changed in an experiment to see how to optimize growth. This is not how nature works. The reality is that there are a multitude of ions interacting biologically with the algal cell. The approach calculates how these multiple ions interact given the physical constraints of each PBR so that their growth is again optimized.

The system also cuts input costs through reusing process water through re-capture, purification, pH/nutrient balancing and then reintroduction. Future cost saving measures will include solar thermal generation coupled to an absorption chiller to provide a more isothermic (same temperature) environment.

An important factor is the combination of the multi-phasic LEDs and biologically optimized nutrient loading. The risk diversification stems from a batch-continuous process, whereby potential contamination or batch failures are contained in 11,355 liter tanks rather than affecting the entire 90,000 liter system. These two key advantages enable the present invention to sustain a competitive advantage by retaining and protecting the intellectual property and maximizing exponential and plateau growth for any given algae.

### Mechanical Separation

The present invention uses separation and clarification utilizing spiralized vertical plates and a self-adjusting interface level technology that allows liquid/liquid clarification within a liquid/solid suspension. One such centrifuge is sold by Evodos manufactured in the Netherlands.

This system will de-water the algae to manageable 3% extracellular water content [versus 30% for traditional centrifuge technology] which will dramatically reduce shipping costs.

### Harvest Enhancement

The present invention has shown that the harvest of grown algae is significantly increase over other known methods and processes.

### BRIEF DESCRIPTION OF THE SEVERAL VIEWS OF THE DRAWING

FIG. 1 is a flow chart showing the seed process of the present invention.

FIG. 2 is a flow chart showing the incubation process and a continuation from FIG. 1.

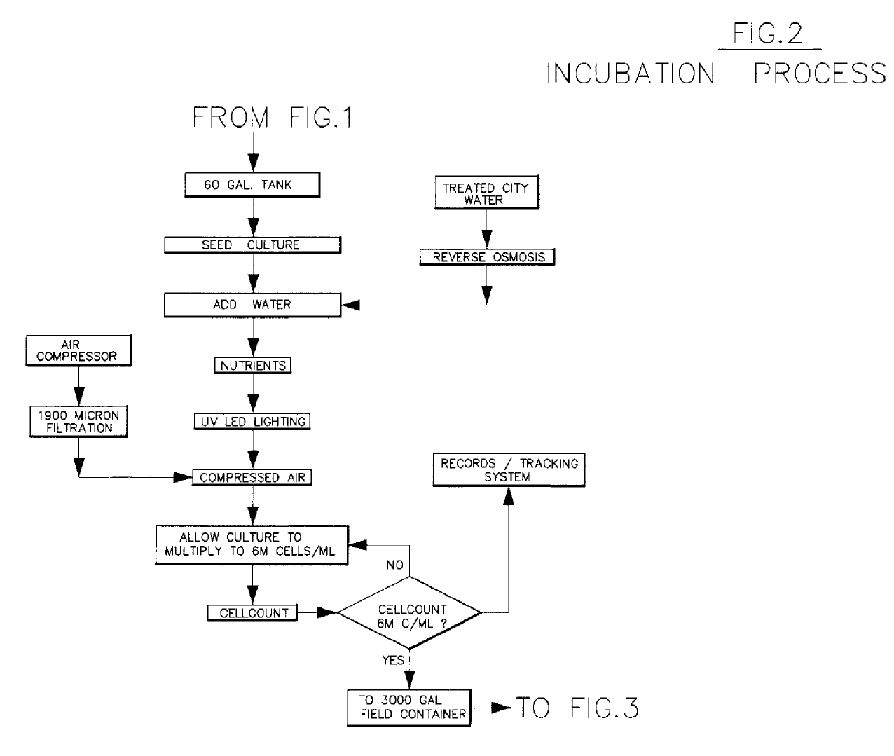

FIG. 3 is a flow chart showing the production process of the present invention.

FIG. 4 is a flow show showing the finished process from the present invention.

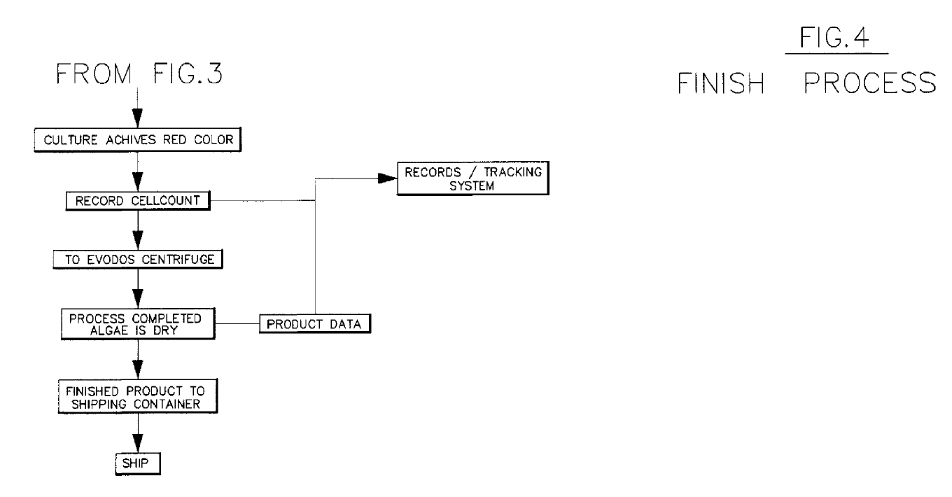

### DETAILED DESCRIPTION OF THE PREFERRED EMBODIMENT

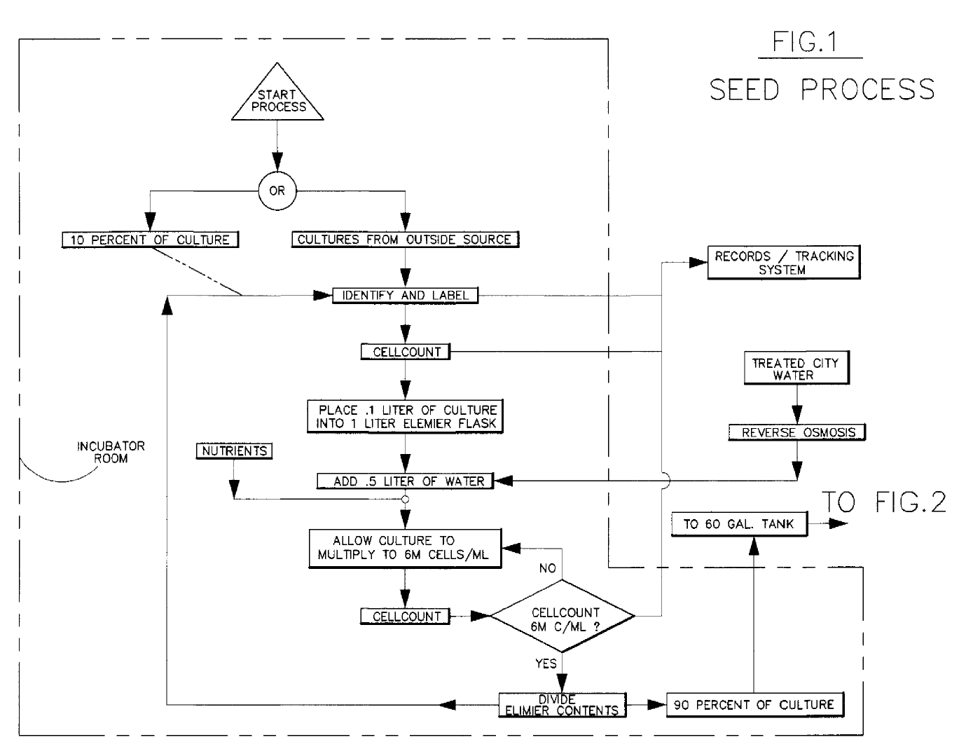

The present invention relates to a closed loop continuous process for algae cultivation and subsequent manufacturing. The system of the present invention begins with a process by which algae are grown. Algae are grown in the present invention as detailed in the beginning of the process shown in FIG. 1. Cultures are initially prepared. Cultures may be either from an outside source or prepared specific for the process of the present invention. In one embodiment the process begins with identification and labeling of a particular set of culture. The culture is identified and an accurate cell count is taken. In one embodiment of the present invention, one hundred milliliters of ten percent culture are placed in a one liter flask. Six hundred milliliters of purified water are added along with appropriate nutrients. The culture is allowed to multiply to a level of six million cells per milliliter. Once the cell count is verified, the culture is then transferred to a second incubation tank. In one embodiment, the incubation tank is a 228 liter incubation tank. Water is added to this 228 liter cylinder, further nutrients and the tank is constructed and arranged to be fitted with natural light through fiber optics and LED lighting. Further chilled compressed air subject to filtration is introduced into the tank.

Although in one embodiment air has been chilled, this is due to the relatively warm ambient air in which this process has been invented. It has been found that the air should be between 70 and 78 degrees Fahrenheit and preferably between 70-75 degrees.

In an environment where the ambient air is below 70 degrees, it may be necessary to heat the air.

In one embodiment, the compressed air is filtered through sub-micron filtration system. The culture is then allowed to subsequently multiply to a level of six million cells per milliliter. A cell count is taken to verify the desired cell growth amount. Once this amount is reached, the subsequent mixture is transferred to a production tank.

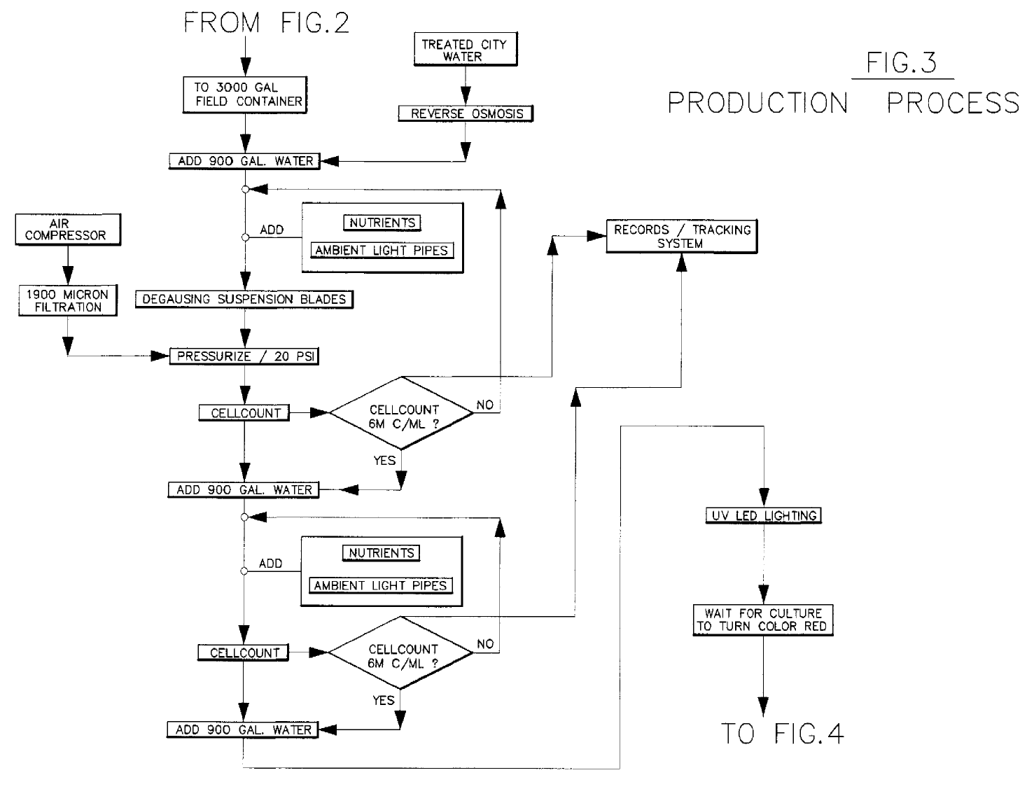

In one embodiment, the production tank is an 11,355 production vessel. As detailed in FIG. 3, 3,408 liters of water are added to the cell mixture transferred from the prior 228 liter cylinder. This will be referred to now as the production mixture. To this production mixture nutrients are and air is introduced in a manner similar to the manner in the incubation process. Nutrients are added and the tank is further exposed to ambient light. Cell count is allowed to reach six million cells per milliliter and as in the incubation process; the production process is further accelerated by the use of natural light through fiber optic and LED light. Once the desired cell count is reached this mixture is transferred to a finishing procedure.

In a preferred embodiment of the present invention a cell growth culture is initiated on a laboratory level in which inert beads are placed within a laboratory flask. It has been surprising found that presence of the inert beads provide between a five to thirty percent accelerated growth within a three day time period of beads and nutrient being added to the flask. After this three to nine day period the initial cell growth medium is transferred to a larger flask wherein said larger flask is between seven to twelve times of greater volume than the initial seeding flask. The algae growth is allowed to continue until there is a six million cell count before further transfer. Six million has been experimentally determined to be an optimal number for transfer. The initial flask is preferably a 1 liter flask in which algae cell growth is allowed to reside for three to six days. In a preferred embodiment the second flask is a 1 liter flask in which cell growth is again subsequently allowed to continue for approximately three to six days. After this three to six day period in the 1 liter flask, the algae cells are transferred to a 228 liter cylinder and further allowed to grow for six to nine days and then transferred as per the flow charts proceeds to a 11,355 liter vessel. It has been discovered that a combination of ambient light through fiber optics and light provided by light emitting diodes (LED) significantly accelerates the growth rate of algae when compared to ambient light alone. Algae growth continues until such time is determined optimum for algae harvest.

Prior to harvest the algae growth has stress placed upon it. Food and nutrients are withheld, and additional water is added. Further, concurrent with the additional added water ultraviolet light is added in a sustained fashion to stress the algae faster. The present invention further provides that after algae are harvested the water is passed through ultraviolet light, sterilized and used to sterilize the harvest tank.

In one embodiment, the starting algae culture is UTEX 2505 Haematococcus Pluvialis. A commercially available nutrient product containing nitrate, phosphate, and trace minerals is used along with a b-complex mix.

For the initial cultivation we use a 1 liter Erlenmeyer flask. In the flask we put nutrient/vitamin mix along with cells from the UTEX Petri tube.

Plastic, clear, beads that are 3×1.7 mm ribbed with 0.35 mm hole are placed in the flask.

When transferring to the 228 liter cylinder, 90% of the initial cultivation material is moved. Approximately 10% is allowed to remain in the flask, and is used in a refill with RO/UV water, nutrients to repeat the process.

The 228 liter cylinder begins with 72 liters of RO/UV water, nutrient/vitamin mix and a natural absorbent and allowed to mix while a pH correct to the medium of around 7.2-7.5 is performed.

After the adjustments, approximately 90% of initial cultivation from 6 flasks is added to the 228 liter cylinder.

When cell count is around 6 million per ml, approximately 72 liters of RO/UV water, nutrient/vitamin mix and a natural absorbent and allowed to mix while a pH correct to the medium of around 7.2-7.5 is performed.

This process is allowed to continue again until cell count is approx. 6 million per ml. The process of adding 72 liters of RO/UV water, nutrient/vitamin mix and a natural absorbent and allowed to mix while a pH correct to the medium of around 7.2-7.5 is performed.

The full cylinder is transferred to 11,355 liter tank.

Sub micron filtered chilled compressed air is gently bubbled into the cylinder.

There are no beads in the final harvest tank.

Regarding the light treatment, the wavelengths of light and types of light sources used in the invention varies from beginning to end. The invention uses a combination of natural and LED light.

In the initial lab flasks, a Lumigrow ES330 LED Grow Light and Parans SP3 fiber optic natural light is used.

During the day, the Parans provides natural daylight (380 nm-750 nm) and at night the Lumigrow provides artificial light (420-720). The light/dark cycle for exposure is around 8/4 and 8/4. Approximately 8 hours of daylight/4 hours of resting dark than 8 hours of LED light/4 hours of resting dark. Intensity of PAR light will be around 400-730 nm.

The cylinders will have LumiBar LED Strip Light, Parans SP3 fiber optic natural light and Caberra G2 ActiveLED-Growbar. The first two have the same spectrum of light as above. The G2 has a 390-780 nm spectrum of light and the same exposure time as in the lab flask stage. Intensity of PAR light will be around 400-730 nm.

The tanks will have the same lights as cylinders until we are ready to stress the algae to cyst and produce astaxanthin/lipids. When there are 6-9 million cells per ml, we will introduce a UVA and UVB wavelength of light in order to stress the algae. The UV spectrum will be 210 and above depending on how much sunlight will be available.

In the harvest tanks, 3,408 liters of RO/UV water, nutrient/vitamin mix and a natural absorbent and allowed to mix while a pH correct is made to the medium to around 7.2-7.5. After the mixture is ready, app 90% of the cylinder is added to the tank.

In the harvest tanks, when cell count is around 6 million per ml, 3,408 liters of RO/UV water, nutrient/vitamin mix and a natural absorbent and allowed to mix while a pH correct is made to the medium to around 7.2-7.5.

Cell count is again allowed to increase to around 6 million per ml, 3,408 liters of RO/UV water, nutrient/vitamin mix and a natural absorbent and allowed to mix while a pH correct is made to the medium to around 7.2-7.5.

Then the algae are stressed and turn red.

The addition of chilled compressed air is by gentle bubbling through 2″ schedule 80 PVC pipes that feeds a diffusing Aero tube. The Aero tubing has thousands of 0.2 micron holes that spans the diameter of the tank and rotates with a gear motor above the tank.

The time in the harvesting tanks will be a maximum of 3 weeks. Algae are harvested using centrifuge at 4200 rpm and cycles for 3 hours before algae are removed.

It should be noted that when transferring from the incubation vessel to the production vessel, the algae mixing must continue in order to not allow the algae to sink to the bottom.

Mixing also allows the algae to evenly flow throughout the tank absorbing nutrients and light. If the algae are mixed to fast, it will cause sheer stress and damage the algae. If it is mixed to slow, algae will sink and not be able to receive light or nutrients and die.

Airlift and bubble columns provide great mixing but require introduction of large amounts of air to mix properly which causes sparger death.

Using a propeller or a pump to mix the algae has to be run at a high rpm which causes shear stress and death.

The present invention incorporates a combination of airlift in bubble columns combined with propeller mixing.

The algae blade will mix anywhere between 6-15 rpm and a aero diffusing tub will run the length of the blade in order to also mix, airlift, while the bubbles will provide a degassing aspect.

A gear motor, rotary coupling, and chain and sprocket will keep the algae blade rotating.

After the process, the algae have been mixed, filled to capacity, stressed, and ready to be centrifuged.

The algae are passed through an Evodos centrifuge system to dewater the algae and the effluent is cleaned and sent back to the tank.

The effluent is run through a 1-3 micron filtering system, U/V light, Ultra filtration, Reverse osmosis system, holding tank with bubbling ozonation, U/V light and then back to the original tank which will also have ozonation.

The water going into the original tank will be fed through a 6 head sprinkler system in order to rinse and sanitize all parts of the tank. The sprinkler system will be in a circular pattern and each sprinkler head will rotate 360 degrees.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. A system for producing algae comprising the steps of:
providing initial algae culture;
placing said algae culture in a vessel;
adding to said vessel inert beads and water to form an algae solution of five to ten percent culture by weight;
growing a first growing of said culture to a desired culture cell count;
transferring said culture to a second vessel whereby said second vessel has an increase in volume of seven to twelve times the volume of said first vessel;
allowing a second growing of said culture to grow to a desired cell culture count;
transferring from said second vessel to an incubation vessel;
adding water and compressed air to said incubation vessel;
providing natural and LED lights to said incubation vessel;
allowing said culture to reach a desired culture cell count;
transferring mixture from said incubation vessel to a production vessel;
adding filtered compressed air, to said production vessel, providing natural and LED light to said production vessel;
allowing said production vessel to incubate until a desired cell count is reached;
harvesting said algae cells from said production vessel.

2. The system of claim 1 wherein said algae culture is freshwater algae.

3. The system of claim 1 wherein said algae culture is from algae in phylum chlorophyta.

4. The system of claim 1 wherein said algae culture is Haematococcus Pluvialis.

5. The system of claim 1 wherein said first growing is to about 6 million cells per ml.

6. The system of claim 1 wherein said production vessel incubation is to a cell count of about 6-9 million cells per ml.

7. The system of claim 1, wherein said compressed air added to each of said incubation and production vessels is provided at 70-78 degrees Fahrenheit.
